SEQUENCE LISTING (62 sequences)

SEQ ID NO: 1 - DNA, 12 bp, Artificial
  zinc finger target sequence
gaagtccaggag

SEQ ID NO: 2 - protein, 7 aa, Artificial
  zinc finger binding domain
RSDNLAR

SEQ ID NO: 3 - protein, 7 aa, Artificial
  zinc finger binding domain
RSDNLRE

SEQ ID NO: 4 - protein, 7 aa, Artificial
  zinc finger binding domain
DRSALSR

SEQ ID NO: 5 - protein, 7 aa, Artificial
  zinc finger binding domain
QSGNLAR

SEQ ID NO: 6 - DNA, 12 bp, Artificial
  zinc finger target sequence
gacccctgggta

SEQ ID NO: 7 - protein, 7 aa, Artificial
  zinc finger binding domain
QSGALAR

SEQ ID NO: 8 - protein, 7 aa, Artificial
  zinc finger binding domain
RSDHLTT

SEQ ID NO: 9 - protein, 7 aa, Artificial
  zinc finger binding domain
TSRDLTE

SEQ ID NO: 10 - protein, 7 aa, Artificial
  zinc finger binding domain
DRANLSR

SEQ ID NO: 11 - DNA, 12 bp, Artificial
  zinc finger target sequence
ggaaagtctccg

SEQ ID NO: 12 - protein, 7 aa, Artificial
  zinc finger binding domain
RSDTLSE

SEQ ID NO: 13 - protein, 7 aa, Artificial
  zinc finger binding domain
NNRDRTK

SEQ ID NO: 14 - protein, 7 aa, Artificial
  zinc finger binding domain
RSDSLSV

SEQ ID NO: 15 - protein, 7 aa, Artificial
  zinc finger binding domain
QNQHRIN

SEQ ID NO: 16 - DNA, 13 bp, Artificial
  zinc finger target sequence
atgggcatcggca

SEQ ID NO: 17 - protein, 7 aa, Artificial
  zinc finger binding domain
QSGSLTR

SEQ ID NO: 18 - protein, 7 aa, Artificial
  zinc finger binding domain
RNDDRKK

SEQ ID NO: 19 - protein, 7 aa, Artificial
  zinc finger binding domain
DRSHLTR

SEQ ID NO: 20 - protein, 7 aa, Artificial
  zinc finger binding domain
RSDALTQ

SEQ ID NO: 21 - DNA, 12 bp, Artificial
  zinc finger target sequence
tgggcatcggca

SEQ ID NO: 22 - DNA, 13 bp, Artificial
  zinc finger target sequence
ccaatgctgcagg

SEQ ID NO: 23 - protein, 7 aa, Artificial
  zinc finger binding domain
RSDTLSA

SEQ ID NO: 24 - protein, 7 aa, Artificial
  zinc finger binding domain
NRSNRIT

SEQ ID NO: 25 - protein, 7 aa, Artificial
  zinc finger binding domain
RSDTLSQ

SEQ ID NO: 26 - protein, 7 aa, Artificial
  zinc finger binding domain
QKATRIT

SEQ ID NO: 27 - DNA, 14 bp, Artificial
  zinc finger target sequence
ccaatgctcaggta

SEQ ID NO: 28 - DNA, 16 bp, Artificial
  zinc finger target sequence
ccaatgctcaggtact

SEQ ID NO: 29 - protein, 7 aa, Artificial
  zinc finger binding domain
QSSDLSR

SEQ ID NO: 30 - protein, 7 aa, Artificial
  zinc finger binding domain
TSSNRKT

SEQ ID NO: 31 - DNA, 12 bp, Artificial
  zinc finger target sequence
tgggacacggcg

SEQ ID NO: 32 - protein, 7 aa, Artificial
  zinc finger binding domain
RSDDLSK

SEQ ID NO: 33 - protein, 7 aa, Artificial
  zinc finger binding domain
RSDTRKT

SEQ ID NO: 34 - protein, 7 aa, Artificial
  zinc finger binding domain
DRSNLSR

SEQ ID NO: 35 - DNA, 12 bp, Artificial
  zinc finger target sequence
aaggtctccgtt

SEQ ID NO: 36 - protein, 7 aa, Artificial
  zinc finger binding domain
TSGSLSR

SEQ ID NO: 37 - protein, 7 aa, Artificial
  zinc finger binding domain
DSSDRKK

SEQ ID NO: 38 - protein, 7 aa, Artificial
  zinc finger binding domain
RLDNRTA

SEQ ID NO: 39 - DNA, 15 bp, Artificial
  zinc finger target sequence
aggaaggtctccgtt

SEQ ID NO: 40 - protein, 7 aa, Artificial
  zinc finger binding domain
RSDHLSA

SEQ ID NO: 41 - protein, 7 aa, Artificial
  zinc finger binding domain
QSGHLSR

SEQ ID NO: 42 - DNA, 15 bp, Artificial
  zinc finger target sequence
aaggtaatttggggt

SEQ ID NO: 43 - protein, 7 aa, Artificial
  zinc finger binding domain
QSSHLTR

SEQ ID NO: 44 - DNA, 12 bp, Artificial
  zinc finger target sequence
gaggaggtggtg

SEQ ID NO: 45 - protein, 7 aa, Artificial
  zinc finger binding domain
RSDALSR

SEQ ID NO: 46 - protein, 7 aa, Artificial
  zinc finger binding domain
RSDALAR

SEQ ID NO: 47 - protein, 7 aa, Artificial
  zinc finger binding domain
RSDNLSR

SEQ ID NO: 48 - protein, 7 aa, Artificial
  zinc finger binding domain
DNNARIN

SEQ ID NO: 49 - protein, 7 aa, Artificial
  zinc finger binding domain
RSDSLSR

SEQ ID NO: 50 - protein, 7 aa, Artificial
  zinc finger binding domain
RKDARIT

SEQ ID NO: 51 - protein, 7 aa, Artificial
  zinc finger binding domain
RSDNLTR

SEQ ID NO: 52 - DNA, 19 bp, Artificial
  zinc finger target sequence
ccaatgctcaggtactggc

SEQ ID NO: 53 - protein, 7 aa, Artificial
  zinc finger binding domain
RSDALSN

SEQ ID NO: 54 - protein, 7 aa, Artificial
  zinc finger binding domain
TSSARTT

SEQ ID NO: 55 - DNA, 1398 bp, Cricetulus griseus
ctagccttaaagacagacagctttgttctagtcagccaggcaagcatatgtaaataaagttcctcagggaactgaggttaaaagatgtatcctggacctgccagacctggccattcacgtaaacagaagattccgcctcaagttccggttaacaacaggaggcaacgagatctcaaatctattacttctaatcgggtaattaaaacctttcaactaaaacacggacccacggatgtcacccacttttccttccccggctccgcccttctcagtactccccaccattaggctcgctactctacctccacttccgggcgcgacacccacgtgccctctcccacccgacgctaaccccgcccctgcccgtctgaccccgcccaccacctggccccgccccgttgaggacagaagaaaccccgggcagccgcagccaaggcggacgggtagacgctgggggcgctgaggagtcgtcctctaccttctctgctggctcggtgggggacgcggtggatctcaggcttccggaagactggaagaaccggctcagaaccgcttgtctccgcggggcttgggcggcggaagaatggccgctagacgcggacttggtgcgaggcatcgcaggatgcagaagagcaagcccgccgggagcgcgcggctgtactaccccgcgcctggagcggccacgccggactgggcggggccggcctggtggaggcggagtctgacctcgtggaggcggggcctctgatgttcaaataggatgctaggcttgttgaggcgtggcctccgattcacaagtgggaagcagcgccgggcgaatgcaatttcgcgccaaacttgggggaagcacagcgtacaggctgcctaggtgatcgctgctgctgtcatggttcgaccgctgaactgcatcgtcgccgtgtcccagaatatgggcatcggcaagaacggagaccttccctggccaatgctcaggtactggctggattgggttagggaaaccgaggcggttcgctgaatcgggtcgagcacttggcggagacgcgcgggccaactacttagggacagtcatgaggggtaggcccgccggctgcagcccttgcccatgcccgcggtgatccccatgctgtgccagcctttgcccagaggcgctctagctgggagcaaagtccggtcactgggcagcaccaccccccggacttgcatgggtagccgctgagatggagcctgagcacacgtgacagggtccctgttaacgcagtgtttctctaactttcaggaacgaattcaagtacttccaaagaatgaccaccacctcctcagtggaaggtaatttggggttaagatgaggattctagggtttgtatgaagcaagatttccaatgcagacgtggaagtgcg

SEQ ID NO: 56 - DNA, 383 bp, Cricetulus griseus
taggatgctaggcttgttgaggcgtggcctccgattcacaagtgggaagcagcgccgggcgaatgcaatttcgcgccaaacttgggggaagcacagcgtacaggctgcctaggtgatcgctgctgctgtcatggttcgaccgctgaactgcatcgtcgccgtgtcccagaatatgggcatcggcaagaacggagaccttccctggccaatgctcaggtactggctggattgggttagggaaaccgaggcggttcgctgaatcgggtcgagcacttggcggagacgcgcgggccaactacttagggacagtcatgaggggtaggcccgccggctgcagcccttgcccatgcccgcggtgatccccatgctgtgccagcctttgc

SEQ ID NO: 57 - DNA, 27 bp, Artificial
  primer
ctagccttaaagacagacagctttgtt

SEQ ID NO: 58 - DNA, 21 bp, Artificial
  primer
cgcacttccacgtctgcattg

SEQ ID NO: 59 - DNA, 22 bp, Artificial
  primer
taggatgctaggcttgttgagg

SEQ ID NO: 60 - DNA, 20 bp, Artificial
  primer
gcaaaggctggcacagcatg

SEQ ID NO: 61 - protein, 5 aa, Artificial
  linker
TGEKP

SEQ ID NO: 62 - protein, 6 aa, Artificial
  linker
TGSQKP

(19) United States
(12) Patent Application Publication
(10) Pub. No.: US 2008/0015164 A1
(43) Pub. Date: Jan. 17, 2008

(54) Methods and compositions for inactivation of dihydrofolate reductase

(75) Inventor: Trevor Collingwood, Novato, CA (US)

Correspondence Address: ROBINS & PASTERNAK, 1731 EMBARCADERO ROAD, SUITE 230, PALO ALTO, CA, 94303, US

(73) Assignee: Sangamo BioSciences, Inc.

(21) Appl. No.: 11/804,222

(22) Filed: May 17, 2007

Related U.S. Application Data
(60) Provisional application No. 60/801,867, filed on May 19, 2006.

(51) Int. Cl. A61K 31/70; C07K 7/00; C12N 15/11; C12N 15/87; C12N 5/06; C12P 21/04

(52) U.S. Cl. 514/44; 435/325; 435/455; 435/69.1; 530/329; 536/22.1

(57) ABSTRACT

Disclosed herein are methods and compositions for inactivating dihydrofolate reductase genes, using fusion proteins comprising a zinc finger protein and a cleavage domain or cleavage half-domain. Polynucleotides encoding said fusion proteins are also provided, as are cells comprising said polynucleotides and fusion proteins.

### CROSS-REFERENCE TO RELATED APPLICATIONS

This application claims the benefit of U.S. provisional patent application No. 60/801,867 (filed May 19, 2006), the disclosure of which is incorporated by reference in its entirety for all purposes.

### STATEMENT OF RIGHTS TO INVENTIONS MADE UNDER FEDERALLY SPONSORED RESEARCH

Not applicable.

### TECHNICAL FIELD

The present disclosure is in the fields of genome engineering, cell culture and protein production.

### BACKGROUND

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, United States Patent Publications 20030232410; 20050208489; 20050026157; 20050064474; 20060188987; 20060063231; and International Publication WO 07/014275, the disclosures of which are incorporated by reference in their entireties for all purposes.

Dihydrofolate reductase (DHFR, 5,6,7,8-tetrahydrofolate:NADP+oxidoreductase) is an essential enzyme in both eukaryotes and prokaryotes and catalyzes the NADPH-dependent reduction of dihydrofolate to tetrahydrofolate, an essential carrier of one-carbon units in the biosynthesis of thymidylate, purine nucleotides, glycine and methyl compounds.

In rapidly dividing cells, the inhibition of DHFR results in the depletion of cellular tetrahydrofolates, inhibition of DNA synthesis and cell death. For example, the DHFR inhibitor methotrexate (MTX) is used as cancer chemotherapy because it can prevent neoplastic cells from dividing. However, the utility of current anti-folate treatments is limited by two factors. First, tumor tissues may rapidly develop resistance to the antifolate, rendering the treatment ineffective. Second, the treatment may be toxic to rapidly dividing normal tissues, particularly to bone marrow or peripheral stem cells.

In addition, DHFR-deficient cells have long been used for production of recombinant proteins. DHFR-deficient cells will only grow in medium supplemented by certain factors involved in folate metabolism or if DHFR is provided to the cell, for example as a transgene. Cells into which a dhfr transgene has been stably integrated can be selected for by growing the cells in unsupplemented medium. Moreover, exogenous sequences are typically co-integrated when introduced into a cell using a single polynucleotide. Accordingly, when the dhfr transgene also includes a sequence encoding a protein of interest, selected cells will express both DHFR and the protein of interest. Furthermore, in response to inhibitors such as MTX, the dhfr gene copy number can be amplified. Accordingly, sequences encoding a protein of interest that are co-integrated with exogenous dhfr can be amplified by gradually exposing the cells to increasing concentrations of methotrexate, resulting in overexpression of the recombinant protein of interest. However, despite the wide use of dhfr-deficient cell systems for recombinant protein expression, currently available DHFR-deficient cell lines do not grow as well as the parental DHFR-competent cells from which they are derived.

Thus, there remains a need for methods and compositions for inactivation of dihydrofolate reducatase (DHFR) to treat folate disorders and to facilitate protein production and overexpression.

### SUMMARY

Disclosed herein are compositions for the partial or complete inactivation of a cellular dihydrofolate reductase (dhfr) gene. Also disclosed herein are methods of making and using these compositions (reagents), for example to inactivate dhfr in a cell for therapeutic purposes and/or to produce cell lines in which a dhfr gene is inactivated.

In one aspect, zinc finger proteins, engineered to bind in a dhfr gene, are provided. Any of the zinc finger proteins described herein may include 1, 2, 3, 4, 5, 6 or more zinc fingers, each zinc finger having a recognition helix that binds to a target subsite in a dhfr gene. In certain embodiments, the zinc finger proteins comprise 4 fingers (designated F1, F2, F3, and F4) and comprise the amino acid sequence of the recognition helices shown in FIG. 1.

In certain embodiments, the disclosure provides a protein comprising an engineered zinc finger protein DNA-binding domain, wherein the DNA-binding domain comprises four zinc finger recognition regions ordered F1 to F4 from N-terminus to C-terminus, and wherein F1, F2, F3, and F4 comprise the following amino acid sequences:

| | | |
|---|---|---|
| F1: | QSGALAR; | (SEQ ID NO: 7) |
| F2: | RSDNLRE; | (SEQ ID NO: 3) |
| F3: | QSSDLSR; | (SEQ ID NO: 29) |
| and | | |
| F4: | TSSNRKT. | (SEQ ID NO: 30) |

In other embodiments, the disclosure provides a protein comprising an engineered zinc finger protein DNA-binding domain, wherein the DNA-binding domain comprises four zinc finger recognition regions ordered F1 to F4 from N-terminus to C-terminus, and wherein F1, F2, F3, and F4 comprise the following amino acid sequences:

| | | |
|---|---|---|
| F1: | RSDTLSE; | (SEQ ID NO: 12) |
| F2: | NNRDRTK; | (SEQ ID NO: 13) |
| F3: | RSDHLSA; | (SEQ ID NO: 40) |
| and | | |
| F4: | QSGHLSR. | (SEQ ID NO: 41) |

In another aspect, fusion proteins comprising any of the zinc finger proteins described herein and at least one cleavage domain or at least one cleavage half-domain, are also provided. In certain embodiments, the cleavage half-domain is a wild-type FokI cleavage half-domain. In other embodiments, the cleavage half-domain is an engineered FokI cleavage half-domain.

In yet another aspect, a polynucleotide encoding any of the proteins described herein is provided.

In yet another aspect, also provided is an isolated cell comprising any of the proteins and/or polynucleotides described herein.

In addition, methods of using the zinc finger proteins and fusions thereof in methods of inactivating dhfr in a cell or cell line are provided. In certain embodiments, inactivating dhfr results in a cell line which can produce recombinant proteins of interest at higher levels (overexpress the protein).

Thus, in another aspect, provided herein is a method for inactivating a cellular dhfr gene (e.g., an endogenous dhfr gene) in a cell, the method comprising: (a) introducing, into a cell, a first nucleic acid encoding a first polypeptide, wherein the first polypeptide comprises: (i) a zinc finger DNA-binding domain that is engineered to bind to a first target site in an endogenous dhfr gene; and (ii) a cleavage domain; such that the polypeptide is expressed in the cell, whereby the polypeptide binds to the target site and cleaves the dhfr. In certain embodiments, the first nucleic acid further encodes a second polypeptide, wherein the second polypeptide comprises: (i) a zinc finger DNA-binding domain that is engineered to bind to a second target site in the dhfr gene; and (ii) a cleavage domain; such that the second polypeptide is expressed in the cell, whereby the first and second polypeptides bind to their respective target sites and cleave the dhfr gene.

In yet another aspect, the disclosure provides a method of producing a recombinant protein of interest in a host cell, the method comprising the steps of: (a) providing a host cell comprising an endogenous dhfr gene; (b) inactivating the endogenous dhfr gene of the host cell by any of the methods described herein; (c) introducing an expression vector comprising transgene, the transgene comprising a dhfr gene and a sequence encoding a protein of interest into the host cell; and (d) selecting cells in which the transgene is stably integrated into and expressed by the host cell, thereby producing the recombinant protein. In certain embodiments, the methods further comprise the step of exposing the host cell comprising the integrated transgene to a DHFR inhibitor (e.g., methotrexate).

In a still further aspect, the disclosure provides a method of treating a subject with a cell proliferative disorder, the method comprising the step of inactivating a dhfr gene according to the method of claim 15 in one or more cells of the subject. In certain embodiments, the cell proliferative disorder is a cancer, for example a leukemia or a lymphoma. The methods may be practiced in vivo or ex vivo.

In any of the cells and methods described herein, the cell or cell line can be a COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), perC6, insect cell such as Spodoptera fugiperda (Sf), or fungal cell such as Saccharomyces, Pischia and Schizosaccharomyces.

### BRIEF DESCRIPTION OF THE DRAWINGS

FIG. 1 is a table depicting exemplary zinc finger designs that bind in a dhfr gene. DNA target sites are indicated in uppercase letters; non-contacted nucleotides are indicated in lowercase letters.

FIG. 2 depicts the target sequences bound by exemplary zinc fingers as described herein and schematically depicts the binding of exemplary zinc finger nuclease (ZFN) pairs within exon 1 of dhfr.

FIG. 3, panels A, B and C, show results of Cel-1 mismatch assays performed on bacterial clones of ZFN-treated cells. The efficacy of each ZFN pair (shown to the left of each panel) is reflected in the total number of cleavage products beneath the parent PCR product.

FIG. 4, panels A, B and C, depict the location of binding of ZFNs 7843 and 7844 within exon 1 of dhfr. FIG. 4A is a schematic of the ZFN dimer. FIG. 4B shows the location of binding sites for ZFNs 7843 and 7844 at the 3′ end of exon 1. FIG. 4C shows the amino acid sequence of the recognition helices of ZFNs 7843 and 7844.

FIG. 5, panels A, B and C, show identification and characterization of DHFR-deficient clones produced using ZFNs. FIG. 5A shows results of a Cel-1 assay. The lane designated “M” is a size marker; lane 1 shows a Cel-1 internal control; lane 2 shows mock-transfected cells; and lanes 3 and 4 show clones from ZFN-treated cells. FIG. 5B shows fluorescent methotrexate (F-MTX) analysis of DHFR expression in initial clones #14 and #15. FIG. 5C shows F-MTX analysis of DHFR expression in subclones of clone #14.

FIG. 6, panels A and B, depict genetic analysis and protection expression of dhfr^{-/-} mutants obtained using ZFNs. FIG. 6A shows partial allelic sequence of clone 14/1, shown as the +1/+2 genotype, and clone 14/7/26, shown as the +2/Δ15 genotype. FIG. 6B shows a Western blot depicting the loss of DHFR protein expression in clones 14/1 and 14/7/26. “CHO-S” refers to wild type cells; “DG44” refers to extract from DHFR-deficient CHO DG44 cells; and “TFIIB” serves as a loading control.

FIG. 7, panels A, B and C, are graphs depicting total cell count in wild-type and exemplary dhfr^{-/-} cell lines obtained using ZFNs with or without hypoxanthine/thymidine (HT) supplement (essential for the growth of cells that do not contain a functional folate metabolic pathway). FIG. 7A shows wild-type cells in which there is little difference in cell count in the presence of absence of HT. FIG. 7B shows cell counts in dhfr^{-/-} cell line designated #14/1. FIG. 7C shows cell counts in dhfr^{-/-} cell line designated #14/7/26. In both FIGS. 7B and 7C, the top line of the graph shows cell counts in the presence of HT and the flat (bottom) line of the graph shows cell counts in the absence of HT. As shown, dhfr^{-/-} cell lines produced by ZFNs exhibit a loss of folate metabolism but no other detectable changes.

FIG. 8 (SEQ ID NO:55) depicts the nucleotide sequence of a 1398 base pair dhfr gene fragment cloned from CHO-S cells (Example 1).

FIG. 9 (SEQ ID NO:56) depicts the nucleotide sequence of the 383 bp PCR product used in Cel-1 mismatch assays (Example 1).

### DETAILED DESCRIPTION

Described herein are compositions and methods for partial or complete inactivation of a dhfr gene. Also disclosed are methods of making and using these compositions (reagents), for example to inactivate a dhfr gene in a target cell. Inactivation of dhfr in a target cell can be used to treat any condition currently treated by folate inhibitors. In addition, compositions and methods as described herein can be used produce cell lines for recombinant protein expression.

The dihydrofolate reductase gene (dhfr) product (DHFR) is required for essential amino acid and nucleotide biosynthesis in all eukaryotic cells. DHFR catalyzes the NADPH-dependent reduction of folate to dihydrofolate and then to tetrahydrofolate. These reduced folates are essential cofactors in the biosynthesis of glycine, purine nucleotides, and the DNA precursor thymidylic acid (Mitchell & Carothers (1986) Mol. Cell. Biol. 6(2):425-440). De novo synthesis of thymidylic acid during S-phase is the major tetrahydrofolate-consuming reaction and thus, activity of this housekeeping gene is particularly important in proliferating cells. Furthermore, the dhfr gene undergoes gene amplification in response to selective pressure, such as the presence of the DHFR-specific inhibitor, methotrexate (MTX). See, Alt et al. (1978) J. Biol. Chem. 253(5):1357-1370; Schimke et al. (1978) Science 202(4372):1051-1055.

Accordingly, DHFR has long been a target for therapeutic intervention. Folate antagonists have been tested as antiinfective, antineoplastic, and anti-inflammatory drugs (Scweitzer et al. (1990) FASEB J. 4(8):2441-2552). The antifolates trimethoprim and pyrimethamine are potent inhibitors of bacterial and protozoal DHFRs, respectively, but are only weak inhibitors of mammalian DHFRs. Methotrexate is the DHFR inhibitor used most often in a clinical setting as an anticancer drug and as an anti-inflammatory and immunosuppressive agent.

The essential role for DHFR in cell growth has also enabled the development of improved mammalian cell-based systems for recombinant protein expression. Considerable effort has focused on enhancing the yield of therapeutic protein production. Early efforts in recombinant DNA technology utilized Chinese hamster ovary (CHO) cell lines in which endogenous DHFR expression had been eliminated or severely reduced by biallelic mutation or deletion at the dhfr locus (Urlaub et al. (1980) Proc. Nat'l. Acad. Sci. USA 77(7):4216-4220; Urlaub et al. (1983) Cell 33(2):405-412). These cells would only grow in medium that was supplemented by components of the folate metabolic pathway and a salvage pathway that circumvented the DHFR deficiency (including, glycine, thymidine and hypoxanthine).

However, the lack of endogenous DHFR can also be overcome by delivering to the cells a plasmid that carries a DHFR expression cassette. This complementation approach allows for the selection of cells in which, on the dhfr^{-/-} genetic background, the dhfr transgene has become stably integrated and expressed. Selection is achieved simply by transferring the cells to medium that is deficient in key substrates of the rescue pathway—usually hypoxanthine and thymidine (Crouse et al. (1983) Mol. Cell Biol. 3(2):257-266). Recombinant protein production processes make use of the fact that exogenous DNA sequences that are closely linked on a plasmid are likely to cointegrate. Thus, by linking the dhfr selection marker to the expression construct of a target protein, the latter will cointegrate with the dhfr transgene gene and stable clones selected for via the dhfr marker.

The copy number of the integrated dhfr marker gene, along with the associated expression cassette of the target recombinant protein, can then be amplified by applying selective pressure in the form of the DHFR inhibitor, methotrexate. Increasing levels of methotrexate selects for only those cells that are able to escape the selective pressure. The ability of cells to survive in the presence of the high levels of methotrexate correlates with increased copy number of the dhfr transgene. During the process of gene amplification, other genes on the plasmid that reside adjacent to the dhfr marker will also become amplified in copy number, thereby increasing the level of their expression products. By this means, an increase in recombinant protein yield can be achieved.

Thus, the methods and compositions described herein provide a highly efficient method for targeted gene knockout that allow for the rapid functional deletion of dhfr without adverse effects on cell growth rate, viability, or other metabolic processes.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119“Chromatin Protocols” (P. B. Becker, ed.) Humana Press, Totowa, 1999.

Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

A “selected” zinc finger protein is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the MRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran. sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

An “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. patent application Ser. Nos. 10/912,932 and 11/304,981 and U.S. Provisional Application No. 60/808,486 (filed May 25, 2006), incorporated herein by reference in their entireties.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression.

“Eucaryotic” cells include, but are not limited to, fungal cells (such as yeast), plant cells, animal cells, mammalian cells and human cells (e.g., T-cells).

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a cleavage domain, the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one ore more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

Zinc Finger Nucleases

Described herein are zinc finger nucleases (ZFNs) that can be used for inactivation of a dhfr gene. ZFNs comprise a zinc finger protein (ZFP) and a nuclease (cleavage) domain.

A. Zinc Finger Proteins

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237.

Enhancement of binding specificity for zinc finger binding domains has been described, for example, in co-owned WO 02/077227.

Selection of target sites; ZFPs and methods for design and construction of fusion proteins (and polynucleotides encoding same) are known to those of skill in the art and described in detail in U.S. patent application Ser. Nos. 10/912,932 and 11/304,981, incorporated by reference in their entireties herein.

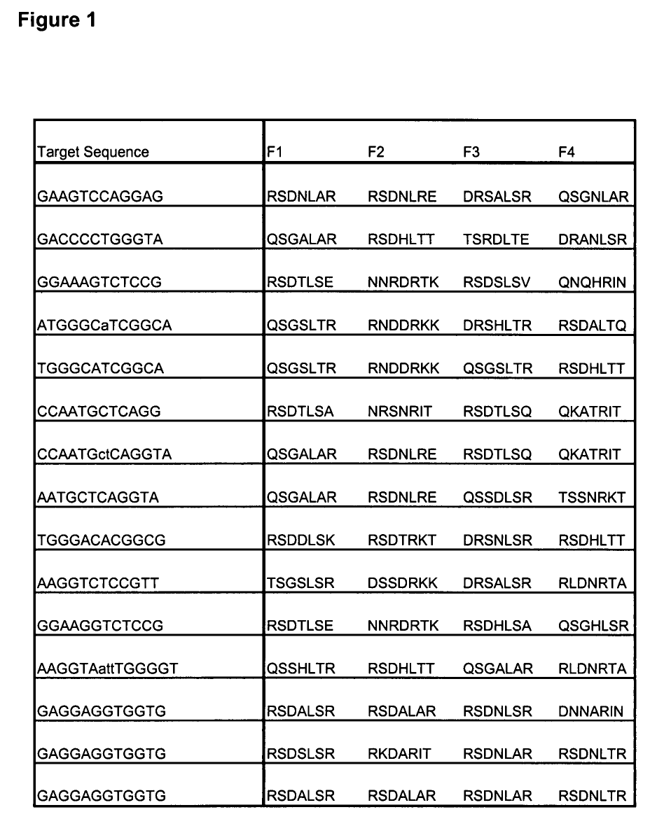

Table 1 describes a number of zinc finger binding domains that have been engineered to bind to nucleotide sequences in the dhfr gene. See, also, FIG. 1. Each row describes a separate zinc finger DNA-binding domain. The DNA target sequence for each domain is shown in the first column (DNA target sites indicated in uppercase letters; non-contacted nucleotides indicated in lowercase), and the second through fifth columns show the amino acid sequence of the recognition region (amino acids −1 through +6, with respect to the start of the helix) of each of the zinc fingers (F1 through F4) in the protein. Also provided in the first column is an identification number for certain proteins.

TABLE 1

Zinc finger nucleases targeted to dhfr

| ZFN name | | | | | |
|---|---|---|---|---|---|
| Target sequence | F1 | F2 | F3 | F4 | F5 |
| ZFN 7832 | RSDNLAR | RSDNLRE | DRSALSR | QSGNLAR | N/A |
| GAAGTCCAGGAG | (SEQ ID | (SEQ ID | (SEQ ID | (SEQ ID | |
| (SEQ ID NO: 1) | NO: 2) | NO: 3) | NO: 4) | NO: 5) | |
| ZFN 7833 | QSGALAR | RSDHLTT | TSRDLTE | DRANLSR | N/A |
| GACCCCTGGGTA | (SEQ ID | (SEQ ID | (SEQ ID | (SEQ ID | |
| (SEQ ID NO: 6) | NO: 7) | NO: 8) | NO: 9) | NO: 10) | |
| ZFN 7834 | RSDTLSE | NNRDRTK | RSDSLSV | QNQHRIN | N/A |
| GGAAAGTCTCCG | (SEQ ID | (SEQ ID | (SEQ ID | (SEQ ID | |
| (SEQ ID NO: 11) | NO: 12) | NO: 13) | NO: 14) | NO: 15) | |
| ZFN 7835 | QSGSLTR | RNDDRKK | DRSHLTR | RSDALTQ | N/A |
| ATGGGCaTCGGCA | (SEQ ID | (SEQ ID | (SEQ ID | (SEQ ID | |
| (SEQ ID NO: 16) | NO: 17) | NO: 18) | NO: 19) | NO: 20) | |
| ZFN 7846 | QSGSLTR | RNDDRKK | QSGSLTR | RSDHLTT | N/A |
| TGGGCATCGGCA | (SEQ ID | (SEQ ID | (SEQ ID | (SEQ ID | |
| (SEQ ID NO: 21) | NO: 17) | NO: 18) | NO: 17) | NO: 8) | |
| 7836 | RSDTLSA | NRSNRIT | RSDTLSQ | QKATRIT | N/A |
| CCAATGCTGCAGG | (SEQ ID | (SEQ ID | (SEQ ID | (SEQ ID | |
| (SEQ ID NO: 22) | NO: 23) | NO: 24) | NO: 25) | NO: 26) | |
| 7837 | QSGALAR | RSDNLRE | RSDTLSQ | QKATRIT | N/A |
| CCAATGctCAGGTA | (SEQ ID | (SEQ ID | (SEQ ID | (SEQ ID | |
| (SEQ ID NO: 27) | NO: 7) | NO: 3) | NO: 20) | NO: 26) | |
| ZFN 7844 | QSGALAR | RSDNLRE | QSSDLSR | TSSNRKT | N/A |
| ccAATGCTCAGGTAct | (SEQ ID | (SEQ ID | (SEQ ID | (SEQ ID | |
| (SEQ ID NO: 28) | NO: 7) | NO: 3) | NO: 29) | NO: 30) | |
| ZFN 7842 | RSDDLSK | RSDTRKT | DRSNLSR | RSDHLTT | N/A |
| TGGGACACGGCG | (SEQ ID | (SEQ ID | (SEQ ID | (SEQ ID | |
| (SEQ ID NO: 31) | NO: 32) | NO: 33) | NO: 34) | NO: 8) | |
| ZFN 7838 | TSGSLSR | DSSDRKK | DRSALSR | RLDNRTA | N/A |
| AAGGTCTCCGTT | (SEQ ID | (SEQ ID | (SEQ ID | (SEQ ID | |
| (SEQ ID NO: 35) | NO: 36) | NO: 37) | NO: 4) | NO: 38) | |
| ZFN 7843 or 9461* | RSDTLSE | NNRDRTK | RSDHLSA | QSGHLSR | N/A |
| aGGAAGGTCTCCGtt | (SEQ ID | (SEQ ID | (SEQ ID | (SEQ ID | |
| (SEQ ID NO: 39) | NO: 12) | NO: 13) | NO: 40) | NO: 41) | |
| ZFN 7847 | QSSHLTR | RSDHLTT | QSGALAR | RLDNRTA | N/A |
| AAGGTAattTGGGGT | (SEQ ID | (SEQ ID | (SEQ ID | (SEQ ID | |
| (SEQ ID NO: 42) | NO: 43) | NO: 8) | NO: 7) | NO: 38) | |
| ZFN 7848 | RSDALSR | RSDALAR | RSDNLSR | DNNARIN | N/A |
| GAGGAGGTGGTG | (SEQ ID | (SEQ ID | (SEQ ID | (SEQ ID | |
| (SEQ ID NO: 44) | NO: 45) | NO: 46) | NO: 47) | NO: 48) | |
| ZFN 7849 | RSDSLSR | RKDARIT | RSDNLAR | RSDNLTR | N/A |
| GAGGAGGTGGTG | (SEQ ID | (SEQ ID | (SEQ ID | (SEQ ID | |
| (SEQ ID NO: 44) | NO: 49) | NO: 50) | NO: 2) | NO: 51) | |
| ZFN 7850 | RSDALSR | RSDALAR | RSDNLAR | RSDNLTR | N/A |
| GAGGAGGTGGTG | (SEQ ID | (SEQ ID | (SEQ ID | (SEQ ID | |
| (SEQ ID NO: 44) | NO: 45) | NO: 46) | NO: 2) | NO: 51) | |
| ZFN 9684 | RSDALSN | TSSARTT | RSDNLRE | QSSDLSR | TSSNRKT |
| ccAATGCTCAGGTACTGgc | (SEQ ID | (SEQ ID | (SEQ ID | (SEQ ID | (SEQ ID |
| (SEQ ID NO: 52) | NO: 53) | NO: 54) | NO: 3) | NO: 29) | NO: 30) |

*ZFNs 7843 and 9461 differ in the linker sequence between F2 and F3. In particular, ZFN 9461 comprises the amino acid sequence TGEKP (SEQ ID NO: 61) between F2 and F3 while ZFN 7843 comprises the amino acid sequence TGSQKP (SEQ ID NO: 62) between F2 and F3.

As described below, in certain embodiments, a four- or five-finger binding domain as shown in Table 1 is fused to a cleavage half-domain, such as, for example, the cleavage domain of a Type IIs restriction endonuclease such as FokI. A pair of such zinc finger/nuclease half-domain fusions are used for targeted cleavage, as disclosed, for example, in U.S. Patent Publication No. 20050064474 (application Ser. No. 10/912,932).

For targeted cleavage, the near edges of the binding sites can separated by 5 or more nucleotide pairs, and each of the fusion proteins can bind to an opposite strand of the DNA target. All pairwise combinations of the designs shown in Table 1 and FIG. 1 can be used for targeted cleavage of a dhfr gene. Following the present disclosure, ZFNs can be targeted to any sequence in a dhfr gene.

B. Cleavage Domains

The ZFNs also comprise a nuclease (cleavage domain, cleavage half-domain). The cleavage domain portion of the fusion proteins disclosed herein can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474 and 20060188987 (application Ser. Nos. 10/912,932 and 11/304,981, respectively) and in U.S. provisional patent application No. 60/808,486 (filed May 25, 2006), the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:1499L”. The engineered cleavage half-domains described herein are obligate heterodimer mutants in which aberrant cleavage is minimized or abolished. See, e.g., Example 1 of U.S. Provisional Application No. 60/808,486 (filed May 25, 2006), the disclosure of which is incorporated by reference in its entirety for all purposes.

Engineered cleavage half-domains described herein can be prepared using any suitable method, for example, by site-directed mutagenesis of wild-type cleavage half-domains (Fok I) as described in U.S. Patent Publication No. 20050064474 (Ser. No. 10/912,932, Example 5) and U.S. Patent Provisional Application Ser. No. 60/721,054 (Example 38).

C. Additional Methods for Targeted Cleavage in DHFR

Any nuclease having a target site in a DHFR gene can be used in the methods disclosed herein. For example, homing endonucleases and meganucleases have very long recognition sequences, some of which are likely to be present, on a statistical basis, once in a human-sized genome. Any such nuclease having a unique target site in a DHFR gene can be used instead of, or in addition to, a zinc finger nuclease, for targeted cleavage in a DHFR gene.

Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

Although the cleavage specificity of most homing endonucleases is not absolute with respect to their recognition sites, the sites are of sufficient length that a single cleavage event per mammalian-sized genome can be obtained by expressing a homing endonuclease in a cell containing a single copy of its recognition site. It has also been reported that the specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66.

Delivery

The ZFNs described herein may be delivered to a target cell by any suitable means. Suitable cells include but not limited to eukaryotic and prokaryotic cells and/or cell lines. Non-limiting examples of such cells or cell lines include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pischia and Schizosaccharomyces.

Methods of delivering proteins comprising zinc fingers are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

DHFR ZFNs as described herein may also be delivered using vectors containing sequences encoding one or more ZFNs. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties.

Conventional viral and non-viral based gene transfer methods can be used to introduce nucleic acids encoding engineered ZFPs in cells (e.g., mammalian cells) and target tissues. Such methods can also be used to administer nucleic acids encoding ZFPs to cells in vitro. In certain embodiments, nucleic acids encoding ZFPs are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids encoding engineered ZFPs include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.).

Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding engineered ZFPs take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications in which transient expression of a ZFP fusion protein is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns etal., Gene Ther. 9:748-55 (1996)).

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a ZFP nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panb cells), GR-1 (granulocytes), and lad (differentiated antigen presenting cells) (see Inaba et al., J. Exp. Med. 176:1693-1702 (1992)).

Vectors (e.g., retroviruses, adenoviruses, liposomes, etc.) containing therapeutic ZFP nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34+ cells, include adenovirus Type 35.

Vectors suitable for introduction of transgenes into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells. Suitable cell lines for protein expression are known to those of skill in the art and include, but are not limited to COS, CHO (e.g., CHO-S, CHO-K1, CHO-DG44, CHO-DUXB11), VERO, MDCK, WI38, V79, B14AF28-G3, BHK, HaK, NS0, SP2/0-Ag14, HeLa, HEK293 (e.g., HEK293-F, HEK293-H, HEK293-T), perC6, insect cells such as Spodoptera fugiperda (Sf), and fungal cells such as Saccharomyces, Pischia and Schizosaccharomyces. Progeny, variants and derivatives of these cell lines can also be used.

Applications

The disclosed methods and compositions can be used for inactivation of a dhfr genomic sequence. Inactivation of a dhfr gene can be achieved, for example, by a single cleavage event, by cleavage followed by non-homologous end joining, by cleavage at two sites followed by joining so as to delete the sequence between the two cleavage sites, by targeted recombination of a missense or nonsense codon into the coding region, by targeted recombination of an irrelevant sequence (i.e., a “stuffer” sequence) into the gene or its regulatory region, so as to disrupt the gene or regulatory region, or by targeting recombination of a splice acceptor sequence into an intron to cause mis-splicing of the transcript.

There are a variety of applications for ZFN-mediated inactivation (knockout) of DHFR. For example, the methods and compositions described herein allow for the generation of new recipient DHFR-deficient cell lines for use in recombinant protein production, including gene amplification systems. Cells as described herein exhibit improved growth characteristics and, accordingly, improved recombinant protein production.

Thus, the DHFR-deficient cell lines described herein overcome a significant drawback faced when using existing DHFR-deficient CHO cells, namely that existing cell lines is exhibit poorer growth characteristics than DHFR-competent parental cell lines, perhaps due to genome-wide damage incurred as a result of the non-specific mutagenic approaches used to destroy the dhfr genes, such as ionizing radiation. By specifically inactivating DHFR in a target cell, the methods described herein provide cells that lack DHFR but exhibit normal growth characteristics.

The methods and compositions described herein also provide a DHFR-based selection strategy that can be used in already highly refined or optimized cell lines, without the risk of reducing the value or effectiveness of the cell line.

In addition, another application of the subject matter disclosed herein is for the therapeutic intervention in any disorder in which an excess of folate is implicated. Non-limiting examples of such disorders include cancerous and non-cancerous cell proliferative disorders such as multiple myeloma, urticaria pigmentosa, systemic mastocytosis, natural killer lymphocyte proliferative disorders (NK-LPD), leukemias, head and neck carcinomas, breast tumors, germ cell tumors, non-Hodgkin's lymphoma, colorectal cancers, gastric cancers, rheumatoid arthritis, psoriasis, autoimmune diseases, and graft-versus-host disease after transplantation.

Many of these disorders are currently treated using folate antagonists (e.g., 5-fluorouracil, methotrexate, aminopterin, trimetrexate, lometrexol, pemetrexed, leucovorin, and thymitaq). In addition, antifolate drugs are also used to treat bacterial infections (trimethoprim), malaria (pyrimethamine), and Pneumocystis carinii infection (trimetrexate with leucovorin). However, as with other drugs used to treat infectious diseases or cancers, the development of resistance limits the effectiveness of these folate antagonists. See, Gorlick et al. (1996) NEJM 335:1041-1048.

Thus, by specifically inactivating a dhfr gene, the compositions and methods of the present disclosure can be readily applied to treatment of any disorder where blocking of folate metabolism is desirable.

### EXAMPLES

### Example 1

### Design and Construction of DHFR-ZFNs

We present here a method for efficient biallelic targeted disruption of the dhfr locus in the widely used recombinant protein production cell line, CHO-S (Invitrogen). The methods exemplified herein make use of one of the cell's innate DNA damage repair pathways: that of non-homologous end joining (NHEJ). By this process, we use engineered zinc finger protein nucleases (ZFNs) to generated double strand DNA breaks within the open reading frame of the dhfr gene. The genetic lesion is then repaired by the imperfect NHEJ process, which often results in mutation at the site of cleavage.

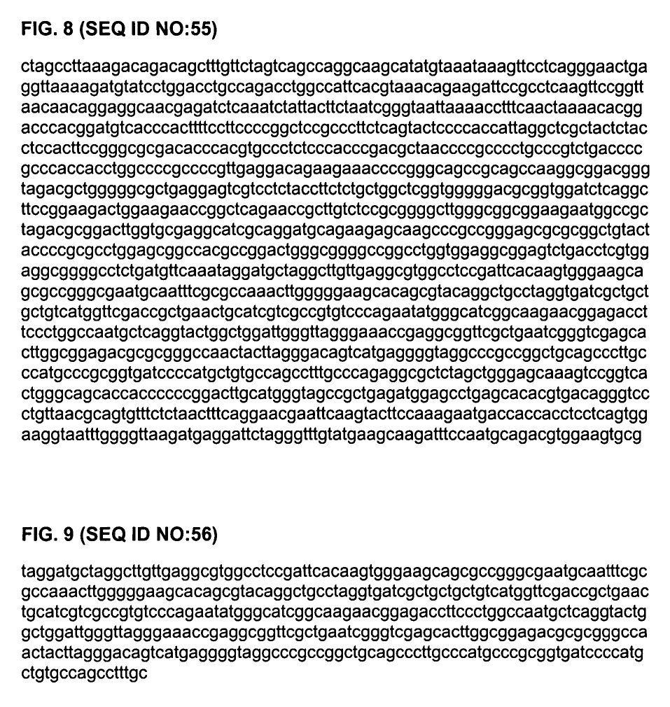

A genomic fragment of the dhfr gene was PCR-cloned from CHO-S cells and sequenced (SEQ ID NO:55; FIG. 8). The sequences of PCR primers used were: 5′ primer 118F-CTAGCCTTAAAGACAGACAGCTTTGTT (SEQ ID NO:57); 3′ primer 107R-CGCACTTCCACGTCTGCATTG (SEQ ID NO:58).

Several pairs of zinc finger nucleases (ZFNs) were designed to recognize sequences within the dhfr genomic fragment. The target sequence for each nuclease is shown in FIG. 1, along with the amino acid sequence of the recognition helices of each of the four zinc fingers.

Plasmids comprising sequences encoding DHFR-ZFNs were constructed essentially as described in Umov et al. (2005) Nature 435(7042):646-651. A set of three nuclease pairs was tested for their capacity to cleave the endogenous CHO dhfr locus at their specified target sites. Adherent CHO-S cells were seeded at 3×10⁵ cells/well in 24-well dishes in DMEM (Invitrogen), 10% FBS γ (JRH BioSciences), Non-Essential Amino Acids (Invitrogen), 8 mM L-Glutamine (Invitrogen), 1×HT supplement (Invitrogen).

The next day, cells were transfected with 100 ng of each ZFN expression plasmid (in pairs)+400 ng pCDNA using Lipofectamine2000™. A portion of each of the treated the cells was harvested after 72 hours and genomic DNA extracted for analysis by the Cel-1 mismatch assay (Example 2).

### Example 2

### Cel-1 Mismatch Assay

The Cel-1 mismatch assay was performed essentially as per the manufacturer's instructions (Trangenomic SURVEYOR™). PCR products were re-annealed by melting at 95° C. followed by slow cooling (2° C./sec to 85° C. and continuing to 25° at 0.1°/sec). To 5 μl of the re-annealed PCR product, 1 μl of Cel-1 endonuclease was added followed by a 20 minute incubation at 42° C. Samples were then run out on a 15% acrylamide gel and visualized using ethidium bromide. Cleavage products indicated the presence of ZFN-mediated mutations in some dhfr alleles at the site of ZFN cleavage.

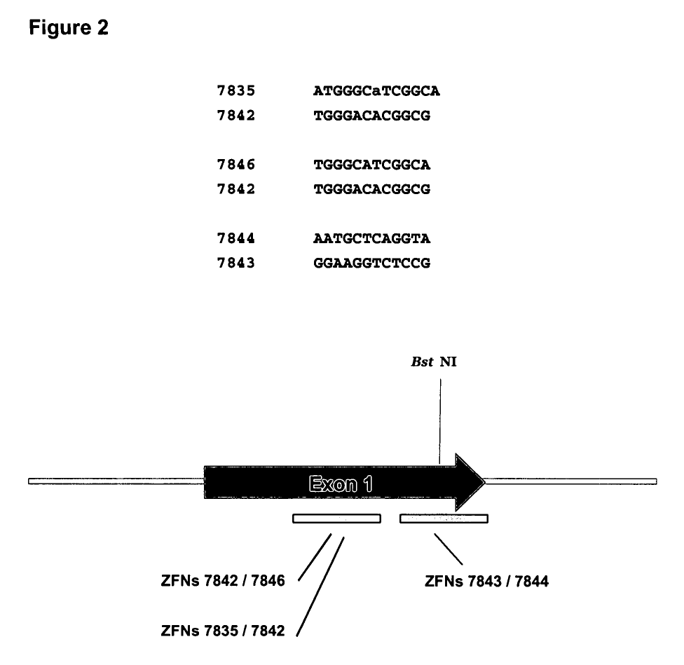

In particular, CHO-S cells were transfected with the three different pairs of ZFNs (7835+7842; 7846+7842; 7844+7843) that each targeted sites within exon 1 of the dhfr gene (FIG. 2). PCR was performed on the genomic DNA extracts from a portion of each treated cell sample, while the remaining cells were kept in culture. The sequence of PCR primers used was: 5′ primer 129F-TAGGATGCTAGGCTTGTTGAGG (SEQ ID NO:59); 3′ primer 130R-GCAAAGGCTGGCACAGCATG (SEQ ID NO:60), generating a 383 bp product from wild type genomic sequence (SEQ ID NO:56; FIG. 9).

The PCR products were then cloned and 96 individual bacterial clones from each sample were resuspended in 10 μl water in a 96-well plate and stored at 4° C. Each colony represented a single allele from the pool of dhfr alleles.

PCR was performed on each colony using primers 129F and 130R. The 96 PCR products from each were pooled into 12 pools of 8 then assayed using the Cel-1 mismatch assay.

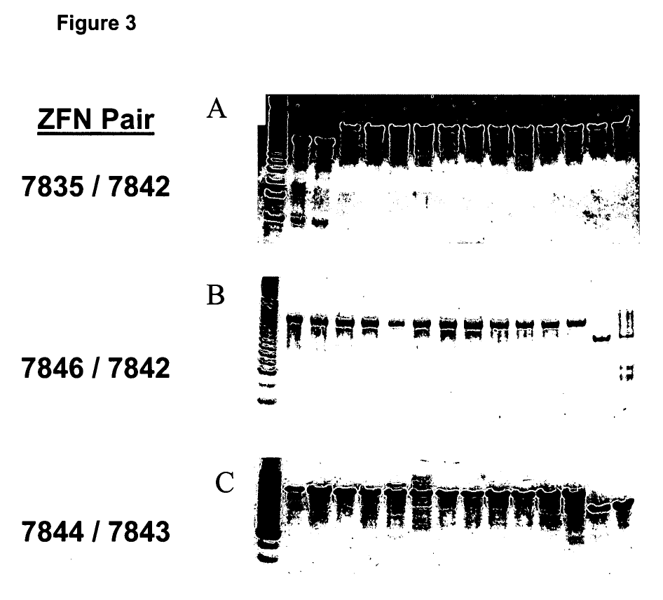

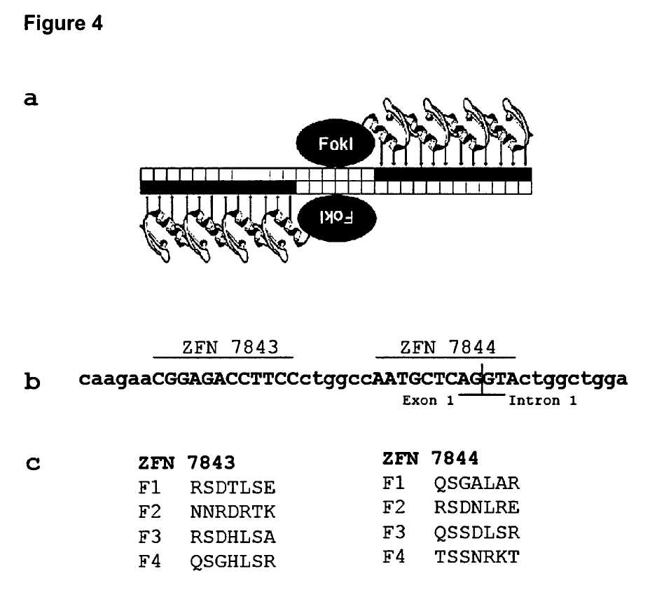

Results of the Cel-1 assay are shown in FIG. 3. As shown, the DNA from cells treated with ZFN pairs 7844+7843 and 7835+7842 exhibited the highest frequency of allelic mutations, as determined by the total number of bands present in all the lanes. ZFN pair 7844+7843 was selected as the lead pair to use for generation of the dhfr knockout cell lines.

In addition, cells were transfected as described above using the ZFN pair 7844+7843, except that at 72 hours post-transfection the ZFN-treated cells were plated into 96-well format at limiting dilution (average of 1 cell/well). As the subclones became confluent they were transferred to 24-well format, at which stage genomic DNA was extracted from each and amplified by PCR using primers 129F and 130R.

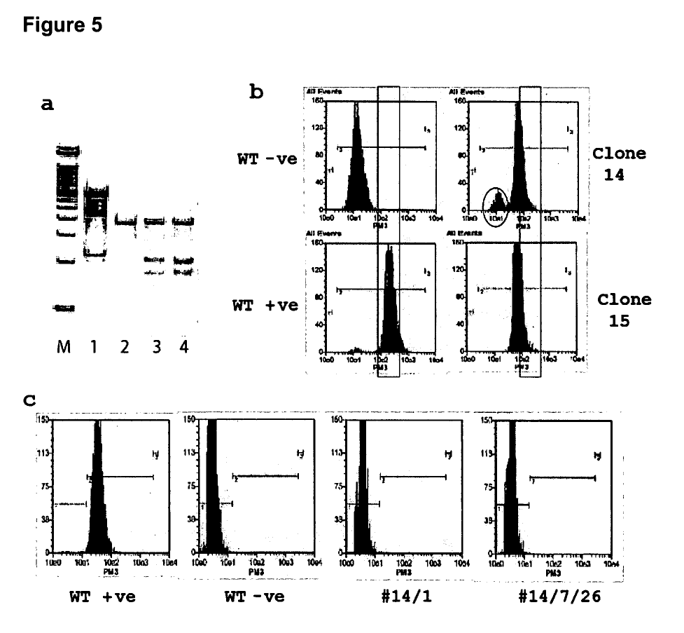

A Cel-1 assay was performed directly on each genomic PCR product. A total of 68 clones were analyzed. Five clones showed the presence of Cel-1 cleavage products. FIG. 5A shows results from the Cel-1 assay for two of these clones, #14 and #15, and indicates the presence of mutant alleles of the dhfr gene.

### Example 3

### Fluorescent Methotrexate (F-MTX) Assay

To demonstrate the loss of functional DHFR expression, clones 14 and 15 were assessed using the fluorescent methotrexate (F-MTX) assay. In this assay, the level of DHFR expression is reflected by the uptake of fluorescent methotrexate into the cells.

Briefly, cells were grown to ˜75% confluency in 24-well plates in adherent serum-containing medium (+HT). The medium was replaced with 500 μl fresh medium containing 10 μM Fluorescein-conjugated methotrexate (F-MTX; Invitrogen/Molecular Probes) and incubated at 37° C. for 2 hours. The medium was removed and replaced with Iml fresh medium without F-MTX and incubated for 30 minutes at 37° C. The medium was removed and the cells washed once with PBS. The cells were trypsinized and resuspended in PBS+1% FBS. Subsequently, 5,000 cells/sample were assessed using a fluorescent cell analyzer (Guava EasyCyte®, Guava Technologies) with excitation at 496 nm, emission at 516 nm.

FIG. 5B shows that for both clones 14 and 15, the major fluorescence peak corresponded to a level of fluorescence (DHFR expression) approximately half that of the wild type positive control, consistent with the possibility that these cells may contain only a single functional allele of the dhfr gene. In addition, clone 14 also showed a small population that exhibited only background fluorescence, suggesting a complete knockout of DHFR expression. Clone 14 was therefore further subcloned and the resulting isolates screened by the F-MTX assay.

FIG. 5C shows F-MTX assay results on two resulting subclones, 14/1 and 14/7/26, both of which fail to take up F-MTX, suggesting that they are devoid of functional DHFR.

### Example 4

### Sequencing and Western Blot Analysis

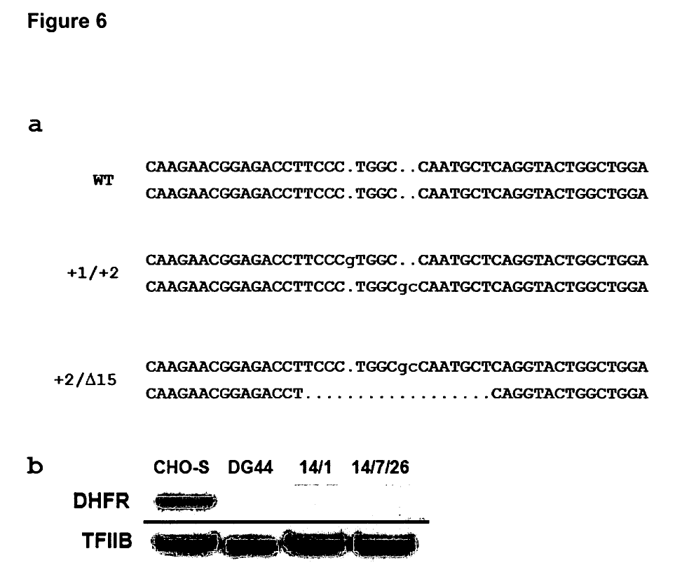

Putative dhfr^{-/-} clones were then analyzed at the genetic level. Genomic PCR products (primers 129F and 130R) were cloned and sequenced. Both clones showed the presence of mutations at the site of nuclease cleavage (FIG. 6A). Clone 14/1 was a compound heterozygous mutant in which one allele contained a single basepair insertion at the cleavage site, while the other allele contained a 2 basepair insertion. Both mutations result in a shift in reading frame that gives rise to multiple stop codons.

Clone 14/7/26 also contained the same 2 basepair insertion in one allele, but the other allele contained a 15 basepair deletion that removed five essential codons.

The complete loss of DHFR protein expression in these clones was further confirmed by Western blot analysis of protein extracts from the cells. Cellular lysates from CHO-S, the dhfr-deficient cell line DG44, CHO-S Clone 14/1, and CHO-S clone 14/7/26 cell lines were blotted and probed initially with a primary antibody against DHFR (Santa Cruz Biotechnology, Cat. # sc-14780; diluted 1:200) and an HRP-conjugated secondary antibody (Santa Cruz Biotechnology, Cat. # sc-2020; diluted 1:5000). The blot was developed using ECL Plus Western blotting Detection Reagents (GE Healthcare). The blot was then re-probed with a TIIFB primary antibody (1 :1000) and a HRP conjugated secondary antibody (1:50,000) (Santa Cruz Biotechnology Cat. # sc-225 and sc-2370 respectively) to serve as a loading control.

As shown in FIG. 6B, clones 14/1 and 14/726 did not contain detectable levels of DHFR.

### Example 5

### Folate-Dependent Growth Assay

To demonstrate the functional disruption of DHFR expression and of the folate pathway, the clones were grown for several days in the absence or presence of hypoxanthine/thymidine (HT) supplement. HT is essential for the growth of cells that do not contain a functional folate metabolic pathway. Cells were seeded in multiple wells at 1×10⁵ cells/well in a 12 well dish containing medium with or without HT supplement. Growth and morphology were monitored daily for 1 week.

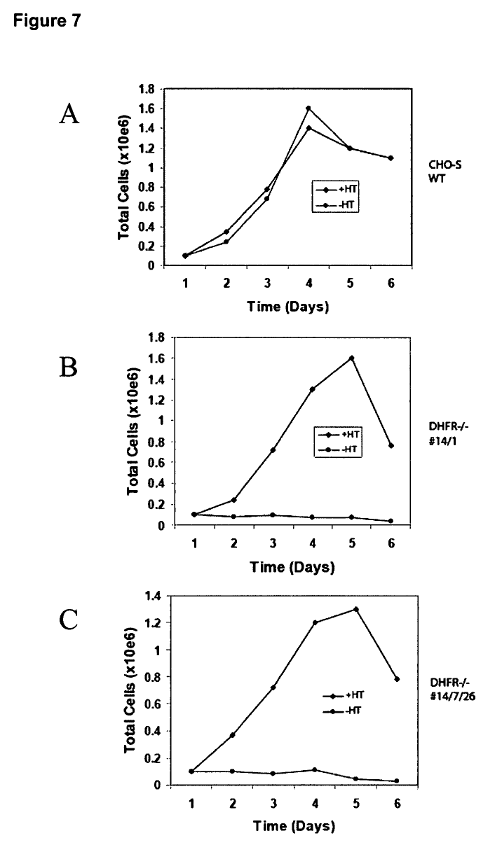

FIG. 7 shows that the growth of both clones requires the presence of HT, whereas wild type CHO-S cells grow equally well in the presence or absence of the supplement. Furthermore, in the presence of HT, the growth of the dhf^{-/-} cell lines shows no significant difference from wild type cells. This indicates that the ZFN-mediated targeting approach has had no detectable effect on the cells other than to knockout the folate metabolic pathway.

These results show the rapid generation of new DHFR-deficient CHO cell line using ZFNs targeted to cleave the dhfr gene within an open reading frame. Illicit DNA repair through the error-prone process of NHEJ at the site of cleavage resulted in functionally deleterious mutations of both alleles and the loss of DHFR protein expression.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A protein comprising an engineered zinc finger protein DNA-binding domain, wherein the DNA-binding domain comprises four zinc finger recognition regions ordered F1 to F4 from N-terminus to C-terminus, and wherein F1, F2, F3, and F4 comprise the following amino acid sequences:

| | | |
|---|---|---|
| F1: | QSGALAR | (SEQ ID NO: 7) |
| F2: | RSDNLRE | (SEQ ID NO: 3) |
| F3: | QSSDLSR | (SEQ ID NO: 29) |
| F4: | TSSNRKT. | (SEQ ID NO: 30) |

2. A protein comprising an engineered zinc finger protein DNA-binding domain, wherein the DNA-binding domain comprises four zinc finger recognition regions ordered F1 to F4 from N-terminus to C-terminus, and wherein F1, F2, F3, and F4 comprise the following amino acid sequences:

| | | |
|---|---|---|
| F1: | RSDTLSE | (SEQ ID NO: 12) |
| F2: | NNRDRTK | (SEQ ID NO: 13) |
| F3: | RSDHLSA | (SEQ ID NO: 40) |
| F4: | QSGHLSR. | (SEQ ID NO: 41) |

3. The protein according to claim 1, further comprising a cleavage domain.

4. The protein according to claim 2, further comprising a cleavage domain.

5. The protein of claim 3, wherein the cleavage domain is a cleavage half-domain.

6. The protein of claim 4, wherein the cleavage domain is a cleavage half-domain.

7. The protein of claim 5, wherein the cleavage half-domain is a wild-type or engineered FokI cleavage half-domain.

8. The protein of claim 6, wherein the cleavage half-domain is a wild-type or engineered FokI cleavage half-domain.

9. A polynucleotide encoding the protein of claim 1.

10. A polynucleotide encoding the protein of claim 2.

11. An isolated cell comprising the protein of claim 1.

12. An isolated cell comprising the protein of claim 2.

13. An isolated cell comprising the polynucleotide of claim 9.

14. An isolated cell comprising the polynucleotide of claim 10.

15. A method for inactivating an endogenous dhfr gene in a cell, the method comprising:
(a) introducing, into a cell, a first nucleic acid encoding a first polypeptide, wherein the first polypeptide comprises:
(i) a zinc finger DNA-binding domain that is engineered to bind to a first target site in an endogenous dhfr gene; and
(ii) a cleavage domain;
such that the polypeptide is expressed in the cell, whereby the polypeptide binds to the target site and cleaves the dhfr.

16. The method of claim 15, wherein the nucleic acid comprises a polynucleotide encoding a protein comprising an engineered zinc finger protein DNA-binding domain, wherein the DNA-binding domain comprises four zinc finger recognition regions ordered F1 to F4 from N-terminus to C-terminus, and wherein F1, F2, F3, and F4 comprise the following amino acid sequences:

| | | |
|---|---|---|
| F1: | QSGALAR | (SEQ ID NO: 7) |
| F2: | RSDNLRE | (SEQ ID NO: 3) |
| F3: | QSSDLSR | (SEQ ID NO: 29) |
| F4: | TSSNRKT. | (SEQ ID NO: 30) |

17. The method of claim 15, wherein the nucleic acid comprises a polynucleotide encoding a protein comprising an engineered zinc finger protein DNA-binding domain, wherein the DNA-binding domain comprises four zinc finger recognition regions ordered F1 to F4 from N-terminus to C-terminus, and wherein F1, F2, F3, and F4 comprise the following amino acid sequences:

| | | |
|---|---|---|
| F1: | RSDTLSE | (SEQ ID NO: 12) |
| F2: | NNRDRTK | (SEQ ID NO: 13) |
| F3: | RSDHLSA | (SEQ ID NO: 40) |
| F4: | QSGHLSR. | (SEQ ID NO: 41) |

18. The method of claim 15, wherein the first nucleic acid further encodes a second polypeptide, wherein the second polypeptide comprises:
(i) a zinc finger DNA-binding domain that is engineered to bind to a second target site in the dhfr gene; and
(ii) a cleavage domain;
such that the second polypeptide is expressed in the cell, whereby the first and second polypeptides bind to their respective target sites and cleave the dhfr gene.

19. A method of producing a recombinant protein of interest in a host cell, the method comprising the steps of:
(a) providing a host cell comprising an endogenous dhfr gene;
(b) inactivating the endogenous dhfr gene of the host cell by the method of claim 15;
(c) introducing an expression vector comprising transgene, the transgene comprising a dhfr gene and a sequence encoding a protein of interest into the host cell; and
(d) selecting cells in which the transgene is stably integrated into and expressed by the host cell, thereby producing the recombinant protein.

20. The method of claim 19, further comprising the step of exposing the host cell comprising the integrated transgene to a DHFR inhibitor.

21. The method of claim 19, wherein the host cell is a CHO cell.

22. A method of treating a subject having a cell proliferative disorder, the method comprising the step of inactivating a dhfr gene according to the method of claim 15 in one or more cells of the subject.

23. The method of claim 22, wherein the cell proliferative disorder is a cancer.
